(19) United States
(12) Patent Application Publication
(10) Pub. No.: US 2010/0189830 A1
(43) Pub. Date: Jul. 29, 2010

(54) Sweet Gum Fruit Extract as a Therapeutic Agent

(76) Inventors: Zhijun Liu, Baton Rouge, LA (US); Peiying Yang, Sugarland, TX (US); Robert A. Newman, Houston, TX (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/600,751

(22) PCT Filed: May 21, 2008
(86) PCT No.: PCT/US08/64303

§ 371 Date: Apr. 12, 2010

Related U.S. Application Data
(60) Provisional application No. 60/939,143, filed on May 21, 2007.

(51) Int. Cl. A61K 36/185; A61P 25/28; A61P 9/10; A61P 9/12; A61P 3/10; A61P 37/06

(52) U.S. Cl. 424/777

(57) ABSTRACT

Sweet gum (Liquidambar styraciflua L., family Hamamelidaceae) fruit extract was discovered to possess potent activities against multiple targets of the PBK (phosphatidylinositide 3-kinase) pathway, especially the PI3K/Akt and mTOR pathways. At a very low concentration of 1.85 μg/ml (IC50), sweet gun extract showed the ability of simultaneously blocking the pathways of PI3K/Akt (upstream), mTOR (mammalian target of rapamycin) (downstream), as well as its downstream protein products S6K and S6. It was also able to block 5-HETE, a lipoxygenase product that contributes to inflammation and activation of PI3K/Akt. The sweet gum fruit extract was prepared with 50% methanol (47:1; raw to extract) and concentrated to an organic fraction (210:1 raw to extract) referred as LIS-100 via reverse-phase column chromatography using a bioassay directed fractionation approach. The extract is a new targeted therapeutic agent for numerous disorders known to be treated by mTOR inhibitors, including cancer, diabetes, obesity, and inflammation.

The benefit of the filing date of provisional U.S. application Ser. No. 60/939,143, filed 21 May 2007, is claimed under 35 U.S.C. §119(e) in the United States, and is claimed under applicable treaties and conventions in all countries.

### TECHNICAL FIELD

An extract from sweet gum (Liquidambar styraciflua L., family Hamamelidaceae) fruit was discovered to be a potent inhibitor against multiple targets of the PI3K (phosphatidylinositide 3-kinase) pathway, especially the PI3K/Akt and mTOR pathways, and to inhibit the proliferation of various cancer cells.

### BACKGROUND ART

Sweet Gum Tree: Sweet gum (Liquidambar styraciflua L., family Hamamelidaceae) is a large aromatic tree native to the Southeastern United States. The spiny pendulous fruits are used as raw plant materials. Although there is no information available on the use of the fruits as medicinal ingredients in the United States, the cousins of sweet gum are used for medicinal purposes. For example, the dried fruit of Liquidambar formosana Hance is an herbal ingredient that appears in the Chinese Herbal Pharmacopoeia as lulutong in Chinese.

Traditionally, sweet gum was used to “dispel wind and remove obstruction from the collaterals, to cause diuresis, and to stimulate menstrual discharge” (Chinese Meteria Medica 1995). Another species of the same genus is Liquidambar orientalis Mill., which has been used to clear mucus congestion and to relieve pain and congestion.

Cinnamic acid and its derivatives as well as triterpenoids are reportedly the main constituents of the bark of sweet gum tree. The sweet gum tree contains cinnamic acid and betulonic acid, 1-methoxy-9-caryolanol, and eudesm-4(14)-ene-1,6-diol. Sweet gum fruits (cones) also accumulate the following triterpene carboxylic acids: 25-acetoxy-3α-hydroxyolean-12-en-28-oic acid; 3β, 25-epoxy-3α-hydroxylup-20(29)-en-28-oic acid, 3β,25-epoxy-3α-hydroxyolean-12-en-28-oic acid; oleanolic acid; butulinic acid; 3-oxohydroxy-olean-12-en-28-oic acid; 3-oxohydroxylup-20 (29)-en-28-oic acid; 3α,25-dihydroxyolean-12-en-28-oic acid; 6β-hydroxy-3-oxolup-20 (29)-en-28-oic acid; 6β-hydroxy-3-oxoolean-12-en-28-oic acid; and 3,11-dioxoolean-12-en-28 oic acid. In examining the anti-tumor activity of sweet gum in a two-stage carcinogenesis assay in mice, 25-acetoxy-3α-hydroxyolean-12-en-28-oic acid and 3β,25-epoxy-3α-hydroxylup-20(29)-en-28-oic acid in the fruits displayed moderate inhibitory activities. The presence of 6β,30-dihydroxy-3-oxolup-20 (29)-en-28-oic acid, 3α-hydroxy-11-oxoolean-12-en-28-oic acid, and massagenic acid G were found in the cones (fruits) of sweet gum. Other compounds that have been isolated from the stem bark of sweet gum include 25-acetoxy-3α-hydroxyolean-12-en-28-oic acid, 3α,25-dihydroxyolean-12-en-28-oic acid, 6β-hydroxy-3-oxolup-20 (29)-en-28-oic acid, and 3,11-dioxoolean-12-en-28 oic acid. It was shown that 25-acetoxy-3α-hydroxyolean-12-en-28-oic acid has strong cytotoxicity against 39 human cancer cell lines, while the other three showed weaker activity. The oral administration of 3β,25-epoxy-3α-hydroxylup-20(29)-en-28-oic acid led to an approximate halving of the tumor incidence and tumor multiplicity in comparison with a control group, which indicated its potential as an anti-tumor agent in UVB-radiation-induced photocarcinogenesis. (Jordan, 1919; Nakajima et al., 1999; Fukuda et al., 2005; Fukuda et al., 2006; Sakai et al., 2004; Fukuda et al., 2006).

Cancer Therapy. Prostate cancer is the second most frequently diagnosed malignancy in men, and prostate cancer ranks as having the highest incidence of any cancer derived from a single organ. Despite the recent decline in the incidence of prostate cancer due to the introduction of prostate-specific antigen (PSA) testing and improved diagnostic modalities, mortality rates have remained unchanged. This is primarily because recurrent prostate cancers that have achieved androgen-independent growth are notoriously difficult to treat. Single cytotoxic agents for the treatment of late-stage prostate cancer, if helpful at all, usually provide no more than a palliative benefit. Thus, a new approach to the management of prostate cancers that have spread beyond the prostate and achieved androgen independence is desperately needed.

Malignant cells, including prostate cancer cells, are often characterized by the up-regulation or constitutive activation of multiple signaling pathways that promote proliferation, inhibit apoptosis, and enable cells to invade and migrate through tissues. Therefore, targeting multiple signaling pathways that are prone to up-regulation in prostate cancer would offer the best prospect for achieving long-term control of this particular tumor. While studies have shown that combinations of chemotherapeutic agents and anti-angiogenic agents at noncytotoxic levels can profoundly suppress tumor growth compared with individual agents alone, an agent that could single-handedly target multiple signaling pathways would be far superior to any single agent, alone or in combination, for the treatment of prostate cancer. (Tortora et al., 2003)

Multiple Signaling Pathways in Cancer: Abberrations of multiple signaling pathways in cancer are common. For example, activation and mutations on PI3 kinase (PI3K), insulin-like growth factor (IGF-1), epidermal growth factor receptor (EGFR), NF-κB, HIF-1α, HSP90, PKA1, COX-2, and 5- and 12-lipoxygenase pathways have been identified in multiple forms of cancer. IGF-1 related molecules are ligands for receptor tyrosine kinases (RTKs) and initiate complex intracellular signaling pathways, such as the PI3K pathway. Direct analysis of cancer samples has led to the discovery of the tumor suppressor gene phosphatase and tensin homologue (PTEN), which has been identified as a hot spot for mutations in glioblastomas, breast and prostate cancer. Loss of PTEN has been correlated with up-regulation of AKT phosphorylation, leading to activation of mTOR activity, and consequently increased activity of ribosomal protein S6 kinase. (McCarty 2004; Chow et al., 2006; Cully et al., 2006)

The PI3Ks are recognized as promising targets for small-molecule inhibitors that have cancer curative potential. This is because increased PI3K activity can be a significant and even critical determinant of the oncogenic cellular phenotype. As demonstrated in FIG. 1, the constitutively active PI3K/Akt signaling axis is critical for tumor cell growth, proliferation, and survival in a variety of cancers, including prostate cancer. PI3K is recruited and activated by cell surface receptors, phosphorylates the D3 hydroxyl of phosphoinositides, and produces phosphatidylinositol (PtdIns)-3 phosphates. Ptdlns (3,4,5,)p3 (PIP3) then binds to the PH domain of Akt, resulting in the translocation of cytosolic Akt to the membrane. The interaction of Akt with its kinase (PDK1 or PDK2) activates Akt kinase activity through the phosphorylation of Thr308 (Akt1) or Ser473 (Akt1), respectively. Activation of Akt then leads to the phosphorylation of multiple downstream substrates that are involved in a variety of physiological events, including cell growth, apoptosis, proliferation, metastasis, and even cell size. A recently identified Akt substrate is tuberin, also called TSC2, which is a tumor suppressor that forms a heterodimeric complex with the TSC1 protein. Akt can then phosphorylate TSC2 at multiple sites, which prevents the substrate from acting as a GAP toward Rheb within cells and allows Rheb-GTP to activate mTOR signaling (13). (Samuels, 2006; Testa et al., 2001; Sarbassov et al., 2005)

mTOR, a 289-kDa serine/threonine kinase belonging to the phosphatidylinositol kinase-related family, is a key regulator of cell growth and proliferation. Its particular role is to regulate ribosomal and cap-dependent translation as well as to influence cell size and autophagy. mTOR activation is a key element of the PI3K/Akt signaling pathway. There are three main downstream messengers of the mTOR kinase, eukaryotic initiation factor 4E-binding protein-1 (4E-BP1), the 40S ribosomal protein S6 kinase (p70S6k, which controls translation and cell cycle progression) and signal transducer and activator of transcription 3 (STAT3; which controls translation and cell cycle progression). It has been well documented that mTOR functions downstream of the PI3K/Akt pathway and is phosphorylated (or activated) in response to stimuli that activate the PI3K/Akt pathway. Interestingly, TSC2, p70S6K, and S6 are also regulated through the Erk pathway. Therefore, mTOR activity can be regulated through both PI3K/Akt and MAPK pathways. Because these are frequently abnormally activated in numerous human maligancies, pathways involving mTOR have become an attractive target for anticancer drug development. In fact, three mTOR inhibitors that are rapamycin derivatives (including Rad001, CCI779 and AP23573) are currently being evaluated in clinical trials. However, several studies have demonstrated that mTOR-specific inhibitors, such as rapamycin, enhance the cell survival pathway, PI3K-Akt activation and e1F4E, in many cell lines as well as tumor biopsy specimens from patients treated with rapamycin derivatives, suggesting the existence of a basal mTOR-dependent feedback mechanism. This feedback activation of Akt by mTOR inhibitors could markedly attenuate the therapeutic effect of an mTOR inhibitor in treating malignant diseases. Activation of Akt kinase in multiple myeloma cells by the mTOR inhibitor was mediated by up-regulation of insulin-like growth factor-I (IGF-I) receptor/insulin receptor substrate-1 (IRS-1) and PI3K/Akt cascade. mTOR activation by insulin initiates a feedback inhibition of PI3K/Akt through p70S6K activation and reduces IRS-1 expression, leading to decreased activity of PI3K/Akt. The inhibition of mTOR was observed to enhance IRS-IGF-1 receptor interactions, inhibit IRS-1 serine phosphorylation and lead to activate PI3K/Akt in multiple cell lines, including multiple myeloma, prostate, breast, and lung cancer cells (FIG. 1). Given the complexity of PI3K/Akt and mTOR cell signaling, including the forward signaling from PI3K to Akt and to mTOR, and the negative feedback from mTOR, and the ability of cells to compensate for loss of function, more than one kinase needs to be inhibited to achieve a significant change in the malignant cell phenotype. On the basis of these observations, dual mTOR and PI3K/Akt inhibitors appear to offer a promising combination therapy. In fact, dual inhibition of PI3K/Akt and mTOR has been shown to be required for shutting down the proliferation of glioma cells, suggesting that agents with a dual inhibitory effect on PI3K and mTOR cell signaling pathways would possess maximum therapeutic efficacy compared with agents that inhibit only one of these kinases. (Vignot et al., 2005; Fingar et al., 2004; Smolewski, 2006; Chambard et al., 2006; Sun et al., 2005; Shi et al., 2005; O'Reilly et al., 2006; Fan et al., 2006)

Mechanistically, PI3K regulates G1 cell cycle progression and cyclin expression in prostate cancer cells through activation of the Akt/mTOR/p70S6k signaling pathway. The growth of prostate tumors in the initial stages is dependent on androgens and is effectively stopped by androgen ablation therapy. However, once the tumor eventually progresses to an androgen-independent phenotype, it turned to be insensitive to hormone withdrawal therapy. Most tumors still continue to express ARs and androgen-related genes, indicating that the AR pathway is still active. Thus, these studies suggest that both PI3K/Akt and downstream mTOR pathways are equally critical in controlling the proliferation of prostate cancer cells, with the mTOR pathway more active in androgen-independent prostate cancer cells, regardless of the status of AR expression. (Gao, N et al., 2003; Xu, Y et al., 2006)

Role of Arachidonic Acid Metabolism in Cancer: In addition to the PI3K/Akt/mTOR pathways, alteration of arachidonic acid metabolism has also been recognized as being important to prostate cancer development. Dietary fat, especially n-6 fatty acids, intake is among the most widely studied risk factor for prostate cancer. Therefore, the most likely mechanism proposed for arachidonic acid-induced cell proliferation in prostate cancer is generation of eicosanoids, which are key mediators of the inflammatory response. Eicosanoids are produced both by tissue cells and by tumor-infiltrating leukocytes and may act as autocrine or paracrine factors. They are synthesized from polyunsaturated fatty acids, with predominantly arachidonic acid released from phospholipids of the cell membrane via the action of phospholipase A2. Eiconsanoids have potent biological activities in cell proliferation and tissue repair, blood clotting, blood vessel permeability, inflammation, and immune cell behavior. There are three known enzymatic pathways involved in eicosanoid metabolism, including the cyclooxygenase (COX-1 and COX-2), lipoxoygenase (LOX, at least 5 known LOX enzymes), and cytochrome P450 enzymes. Eicosanoids fall into three general groups, prostaglandins (PGs), leukotrienes (LTs), and thromboxanes. (FIG. 3). These specific eicosanoids are modulators of tumor cell interactions with certain host components within the context of cancer growth, invasion, and metastasis. (Wang et al., [?]; Higgs et al., 1984; Wallace, 2002; Jiang et al., 2004).

In contrast to lung cancer, in which prostaglandin metabolism is a dominant tumorigenic factor, LOX products appear to plan an important role in prostate cancer etiology. For example, 12-HETE, a 12-LOX product, promotes the proliferation of human prostate cells, as well as that of colon, pancreatic, and breast cancer cells, and plays an important role in cell adhesion and promotion of metastasis. 12-LOX also appears to stimulate angiogenesis in human prostate carcinoma cells. Inhibition of proliferation of PC3 prostate cancer cells was shown to be associated with inhibition of 12-LOX. Additionally, the 5-LOX product, 5-HETE, has been suggested to function as a potent survival factor in human prostate cancer cells. In contrast, 15-LOX-2 and its arachidonic acid metabolite, 15-HETE, were shown to function as tumor suppressors in prostate cancer cells. Thus 12-HETE and 5-HETE can both promote the growth of cancer cells, but 15-HETE is considered a tumor suppressor. (Nie et al., 2001; Nie et al., 2003; Yang et al., 2007; Ghosh et al., 1997; Yang et al., 2003; Subbarayan et al., 2001; Tang et al., 2002)

Implication of mTOR pathway on other diseases. Over the past several years, an increasing number of human diseases have been linked to the dysregulation of mTOR cell signaling pathway, including cancer, diabetes, cardiovascular and neurological disorders. Intriguingly, most of these diseases are due to aberrant hyperactivity of the mTOR pathway, which makes an inhibitor of mTOR potentially effective therapeutics for the treatment of these diseases Inhibitor of mTOR have been proposed to be useful in treatment for the following diseases—including rejection of allografts, autoimmune disorders (e.g., allergic encephalomyelitis, insulin-dependent diabetes mellitus, lupus, adjuvant arthritis, rheumatoid arthritis, psoriasis, and multiple sclerosis), cancer(e.g., prostate cancer, renal cell carcinoma, mantle cell lymphoma, endometric cancers, other cancers sensitive to mTOR inhibitors), diabetes (e.g., both Type 1 and Type 2 diabetes), obesity (e g , inhibition of fat accumulation), cardiovascular diseases (ischemic disease, hypertension, valvular disease, and heart failure), and neurological disorders (e.g., Huntington's, Alzheimer's and Parkinson's diseases). (See Tsang et al., 2006; Hartford et al., 2007)

There are two types of diabetes. Type 1 diabetes is caused by loss of insulin production due to destruction of pancreatic β-cells. Type 2 diabetes is developed when insulin secretion from pancreatic β-cells fails to compensate for the peripheral insulin resistance in skeletal muscle, liver and fat cells. Clinical studies using immunosuppressive regimens containing rapamycin to prevent the rejection of islet transplants have shown some significant efficacy in type 1 diabetes patient. However, there is a special positive role of mTOR in the growth of pancreatic β-cells. Thus, mTOR inhibition needs to be balanced carefully in islet transplantation. (Shapiro et al., 2000)

An mTOR inhibitor (e.g., sweet gum fruit extract) could be potentially useful in management of Type 2 diabetes due to the following reasons: (1) Evidence indicates that insulin resistance might be caused by inhibition of insulin-receptor substrate (IRS) proteins by phosphorylation, which abolishes the signaling transduction pathway from insulin receptor to PI3 kinase. Recent data suggest that sustained activation of mTOR signaling is a crucial event that makes IRS not responsible to insulin. (2) An mTOR inhibitor, such as rapamycin, can restore the sensitivity of IRS to insulin by negative feed-back loop due to mTOR being a downstream of the IRS-PI3K-Akt pathway. (3) It has been demonstrated that S6k1, an effector of mTOR pathway, phosphorylates IRS directly, and resulted in the inhibition of the association of IRS with insulin receptor. (4) More recent research indicated that chronic hyperglycemia can lead to activation of mTOR in pancreatic β-cells. Chronic activation of mTOR could trigger proteosomal degradation of IRS2, which would lead to an increase in apoptosis of β-cells. (Saltie et al., 2001; Shah et al., 2004; Harrington et al., 2004; Briaud et al., 2005)

Taken together, these studies have suggested that activation of mTOR in insulin responsive cells reduces insulin signaling and leads to insulin resistance. On the other hand, persistent activation of mTOR in pancreatic β-cells could contribute to reduction of cell mass and insulin secretion. Therefore, the mTOR inhibitor would have great potential for the treatment of Type 2 diabetes.

In addition, renal enlargement occurs in early phases of human and experimental diabetes and contributes to the later development of overt diabetic kidney disease. The renal enlargement is mainly due to renal cellular hypertrophy. mTOR signaling has been reported to regulate protein synthesis and cellular growth, specifically hypertrophy. It has been suggested that activation of mTOR signaling, especially increased phosphorylation of effector of mTOR, p70S6k, causes renal hypertrophy at early stage of diabetes in an animal model. Intraperitoneal injection of rapamycin markedly attenuated the enhanced phosphorylation of p70S6k and subsequent renal enlargement without any changes in clinical parameters in mice bearing streptozotocin-induced diabetes. Therefore, modulation of mTOR activity might represent a novel approach for diabetic nephropathy. (Sakaguchi et al., 2006)

The most common mode of treatment for early stage of prostate cancer, the growth of which is dependent on androgens, is surgical or pharmacological castration to decrease androgen action. However, this therapy often fails when tumor growth becomes independent of androgen (late stage). Androgen independent prostate cancer tends to progress and metastasize to a point where there is no consensus therapy. Insights into the epidemiology, genetics, and molecular pathogenesis of prostate cancer strongly suggest that PI3K/Akt and mTOR activation play important roles in the progression of prostate cancer to an androgen-independent state. As such, mTOR inhibitors are showing promise as useful targeted chemotherapeutic agents in many malignancies, including late stage prostate cancer. However, unexpected feedback activation of Akt by mTOR inhibitors has become a barrier for the effective use of this class of drugs. Suppressing both Akt and mTOR pathways is highly desirable but no such an agent has previously been identified.

### DISCLOSURE OF INVENTION

Sweet gum (Liquidambar styraciflua L., family Hamamelidaceae) fruit extract was discovered to be a potent inhibitor against multiple targets of the PI3K (phosphatidylinositide 3-kinase) pathway, especially the PI3K/Akt and mTOR pathways. At a very low concentration of 1.85 μg/ml (IC₅₀), a purified fraction of sweet gun extract (named “LIS-100”) simultaneously blocked the pathways of PI3K/Akt (upstream) and mTOR (mammalian target of rapamycin) (downstream), as well as the mTOR downstream protein products S6K and S6. This extract also blocked 5-HETE, a lipoxygenase product that contributes to inflammation and activation of PI3K/Akt. The initial sweet gum fruit extract was prepared with 50% methanol (47:1; raw to extract) and concentrated to an organic fraction (210:1 raw to extract). The extract was fractionated and the specific fraction, referred to as LIS-100, was identified using reverse-phase column chromatography using a bioassay directed fractionation approach. The LIS-100 extract was shown to be fifteen times more effective than could be explained on the basis of a known active single component of sweet gum fruits. The extract contains several components that appear to act synergistically. LIS-100 is a good candidate for a therapeutic agent in diseases, including cancer, diabetes, obesity, and inflammation. At concentrations as low as 0.5 μg/ml, LIS-100 almost totally shut down the expression of effectors of the mTOR pathway, yet did not cause activation of Akt. In fact, it simultaneously inhibited phosphorylation of Akt in PC3 cells, suggesting that LIS-100 is dual inhibitor of PI3K/Akt and mTOR pathways. Because of this property sweet gum extract is advantageous over the current therapy and presents a new opportunity in the treatments of PI3K mediated diseases including, but not limited to, late-stage prostate cancer.

In particular, LIS-100 can be used for targeted cancer therapy for late stage prostate cancer, since inhibiting both the PI3K/Akt and mTOR pathway can control late stage prostate cancer, while inhibiting a single target (e.g., an mTOR inhibitor) has not been a successful approach.

In addition, the sweet gum extract (LIS-100) as a potent inhibitor of mTOR will be useful in treatment for diseases known to be affected by inhibitors of the mTOR pathway—including rejection of allografts, autoimmune disorders (e.g., allergic encephalomyelitis, insulin-dependent diabetes mellitus, lupus, adjuvant arthritis, rheumatoid arthritis, psoriasis, and multiple sclerosis), cancer (e.g., prostate cancer, renal cell carcinoma, mantle cell lymphoma, endometric cancers, other cancers sensitive to mTOR inhibitors), diabetes (e.g., both Type 1 and Type 2 diabetes), obesity (e.g., inhibition of fat accumulation), cardiovascular diseases (ischemic disease, hypertension, valvular disease, and heart failure), and neurological disorders (e.g., Huntington's, Alzheimer's and Parkinson's diseases). (See Tsang et al., 2006; Hartford et al., 2007)

### BRIEF DESCRIPTION OF DRAWINGS

FIG. 1 illustrates a schematic representation of the PI3K pathway and indicates the possible target compounds of LIS-100, an active anticancer fraction of sweet gum. The dashed line shows the most plausible mechanism by which the mTOR inhibitor rapamycin activates Akt.

FIG. 2 illustrates a schematic representation of the possible signaling mechanisms leading to cell cycle progression in the two prostate cancer cell lines, LNCaP (androgen dependent) and C4-2 (androgen independent) cells.

FIG. 3 illustrates a schematic representation of the arachiodonate metabolism cascade known to be important to prostate cancer development.

FIG. 4A illustrates the antiproliferative activity of sweet gum extract (LIS-F) against various human cancer cell lines, including human prostate (PC3), colon (HCT116), pancreatic (BxPC3, Panc-1, AsPC-1), and non-small cell lung cancer cells (A549 cells).

FIG. 4B illustrates the antiproliferative activity of sweet gum extract (LIS-F) and four fractions of LIS-F (LIS-00, LIS-20, LIS-50, and LIS 100) against the human prostate cancer cell line (PC3 cells).

FIG. 4C illustrates the antiproliferative activity of the sweet gum extract (LIS-100) on PC3 (prostate cancer), LNCaP (prostate cancer), HCT116 (pancreatic cancer), DU145 (prostate cancer) and Panc-1 cells (pancreatic cancer).

FIG. 5 illustrates the chromatographic fingerprint of sweet gum fruit extracts, including the crude extract (LIS) and various fractions of the crude extract (LIS-00, LIS-20, LIS-50, and LIS-100).

FIG. 6A illustrates the effect of LIS-F on the cell cycle of prostate cancer (PC3) cells.

FIG. 6B illustrates the effect of the sweet gum extract, LIS-100, on the cell cycle of prostate cancer (PC3) cells.

FIG. 6C illustrates the effect of the sweet gum extract, LIS-100, on apoptosis of prostate cancer (PC3) cells.

FIG. 7 illustrates the change in eicosanoid metabolism in prostate cancer (PC3) cells treated with different concentrations of LIS-F and LIS-100 (25 to 100 μg/ml).

FIG. 8A illustrates the change in cell signaling pathways in prostate cancer (PC3) cells treated with several high concentrations of LIS-100 (0, 5, 10, 25, and 50 μg/ml).

FIG. 8B illustrates the change in cell signaling pathways in prostate cancer (PC3) cells treated with several low concentrations of LIS-100 (0, 0.5, 1, 5, 10 μg/ml).

FIG. 8C illustrates the change in cell signaling pathways in prostate cancer (PC3) cells treated with several high concentrations of LIS-F (0, 5, 10, 25, and 50 μg/ml).

FIG. 9 illustrates the effect of sweet gum extract (LIS-100, 2 μg/ml) on autophagic cell death in prostate cancer (PC3) cells for treatment at various time periods, as measured by the expression of the hallmark autophagic protein, LC3-II.

FIG. 10 illustrates the effect of sweet gum extract (LIS-100 at 1, 5, and 10 μg/ml) and rapamycin and a control on activity of PI3-kinase in prostate cancer (PC3) cells, as measured by the reduction in phosphorylation of the protein, pGSK-3.

FIG. 11 illustrates the effect of sweet gum extract (LIS-100 at 5, 10, and 25 μg/ml) and rapamycin on activity of mTOR in prostate cancer (PC3) cells relative to the control activity.

FIG. 12 illustrates the effect of treatment with sweet gum extract (LIS-100, at 50 mg/kg (IP) or 250 mg/kg (oral)), rapamycin, or controls on the size of a mouse xenograft tumor (human PC3) after three weeks of treatment.

FIG. 13A illustrates the chromatographic fingerprint of the crude sweet gum extract (LIS-F) developed at 254 nm.

FIG. 13B illustrates the chromatographic fingerprint of the sweet gum extract (LIS-100) developed at 254 nm.

FIG. 14A illustrates the chromatographic fingerprint of the crude sweet gum extract (LIS-F) developed at 203 nm, illustrating the presence of unknown triterpenoids.

FIG. 14B illustrates the chromatographic fingerprint of the sweet gum extract (LIS-100) developed at 203 nm, illustrating the presence of unknown triterpenoids.

FIG. 15 illustrates a comparison between the chromatographic fingerprint of the sweet gum extract (LIS-100) developed at 203 and 254 nm, and one of ellagic acid and 25-acetocy-3α-hydroxyolean-12-en-28-oic acid, an oleanane-type triterpeneoid, developed at 254 nm.

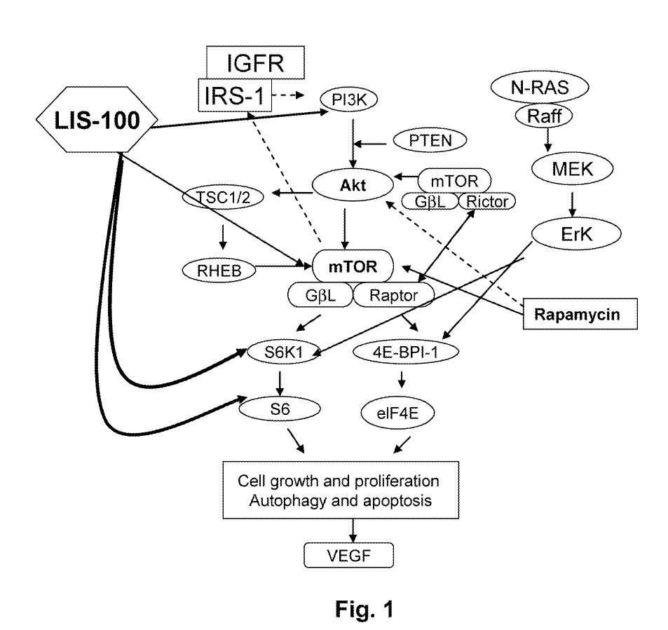

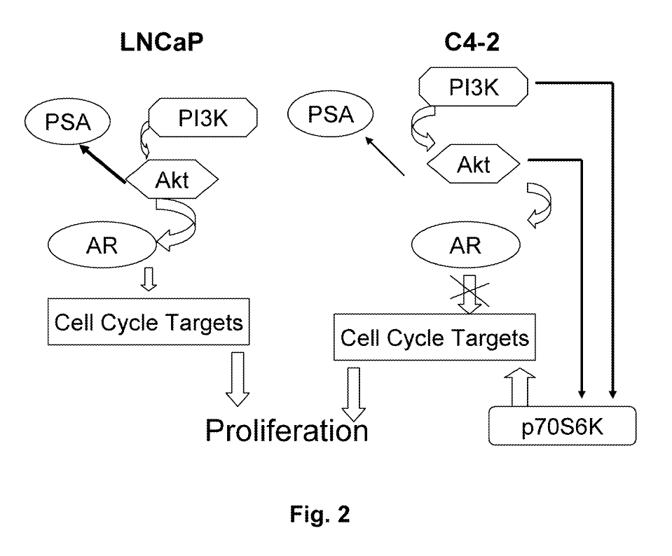

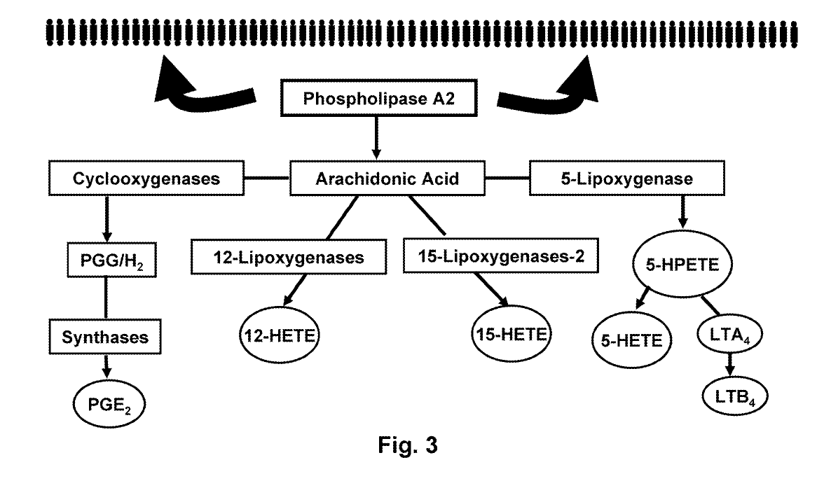

### MODES FOR CARRYING OUT THE INVENTION

### Example 1

Materials and Methods

Plant material extraction and fractionation. Sweet gum (Liquidambar styraciflua L.) fruit was collected in season in Louisiana, oven-dried, and ground to pass through a 6-mm sieve. The ground particles were extracted using 50% aqueous methanol at a raw:solvent ratio of 1:10 w/v at 60° C. for 4 hr. After filtration through Whatman # 4 filter papers (>20 μm), the organic solvent was evaporated from the filtrate, and the filtrate was freeze-dried to powder. This powder is the crude extract LIS-F. LIS-F was then dissolved in water at 1:33 ratio (w/v). The aqueous solution was subjected to column chromatographic separation by C18 sorbent material using an Isco Companion Flash Chromatography unit with online UV detection. Sequential elution with increasing solvent (0%, 20%, 50%, and 100% aqueous methanol) yielded four fractions, named LIS-00, LIS-20, LIS-50 and LIS-100.

Chromatography of Extracts. An analytical method for determining the chromatographic fingerprint of the sweet gum extracts was been established. This method showed good separation of major components and was used to monitor the sub-fractionation and isolation processes. The method used an HPLC system (Waters Delta 600, Waters Co., Milford, Mass.) consisting of a solvent delivery pump unit, an autosampler (Waters 717 plus), a UV-Vis diode array detector (Waters 2996 Photodiode Array Detector, 190 to 800 nm) coupled with an EMD 1000 Mass Detector (Waters), and an evaporative light-scattering detector (Waters 2420 ELSD). This system can analyze both analytical and preparative separations. Separation of the fractions was performed using a Symmetry C18 column (5.0 μm, 150×4.6 mm I.D.; Waters) with a guard cartridge (5.0 μm, 20×3.9 mm I.D.), with a flow rate of 1.0 ml/min and a detection wavelength of 254, 230 or 203 nm, or any wavelength in the range of 200-400 nm. The mobile phase was as follows: 0-5 min, 10% MeOH; 5-28 min, gradient to 80% MeOH; 28-30 min, gradient to absolute MeOH until 40 min; 40 min, 10% MeOH to end.

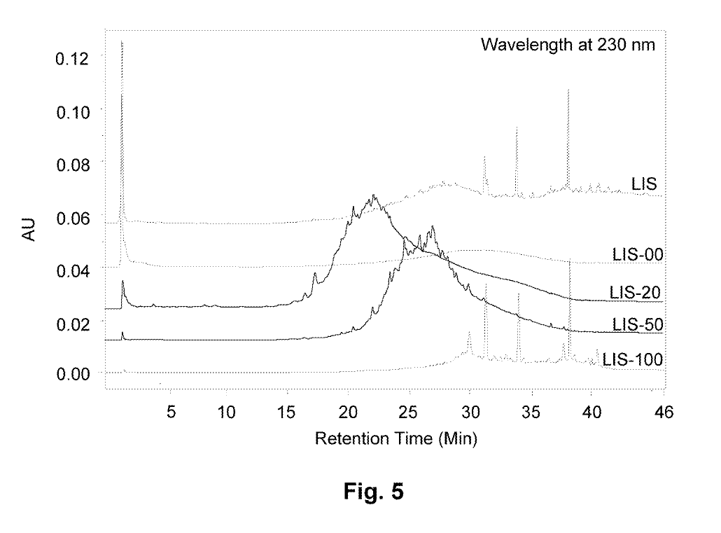

A chromatographic fingerprint for LIS-100 was generated using LC-MS instrumentation to provide specific UV spectrum, MS spectra, and retention time information for each peak. This chromatographic fingerprint was used as a template for controlling the quality of LIS-100 extracts. (See FIGS. 5, 13B, 14B, and 15)

### Example 2

Antiproliferative Activity of Extracts of Sweet Gum

The crude sweet gum extract (47:1, LIS-F) and the purified extract (LIS-100) were prepared from the fruit with 50% methanol extraction as discussed above. LIS-F inhibited the proliferation of multiple cell lines, including human prostate cancer (PC3, LNCaP, and DU145), human colon HCT116, human pancreatic cancer (PANC-1, BxPC3, and AsPC-1), and human non-small cell lunch cancer A549 cells. All of these human cancer cell lines were purchased from the American Type Culture Collection (Manassas, Va.) and maintained in a humidified atmosphere containing 5% CO₂ at 37° C. Cell lines derived from different epithelial origins were routinely cultured in tissue culture medium (Invitrogen Corp., Grand Island, N.Y.) (Table 1) supplemented with 10% heat-inactivated fetal bovine serum ([FBS] Hyclone Laboratories Inc., Logan, Utah), 50 IU/ml penicillin and 50 μg/ml streptomycin, and 2 mM L-glutamine from GIBCO (Invitrogen). (See Table 1).

TABLE 1
Description of human cancer cell phenotype and cell culture medium

| Name of cells | Phenotype description | Cell culture medium |
|---|---|---|
| PANC-1 | Human pancreatic | DMEM/10% FBS |
| | carcinoma | |
| BXPC3 | Human pancreatic | RPMI 1640/10% FBS/sodium |
| | adenocarcinoma | pyruvate (NaP) |
| AsPC-1 | Human pancreatic | RPMI 1640/10% FBS/sodium |
| | adenpcarcinoma | pyruvate (NaP) |
| HCT 116 | Colon carcinoma | RPMI 1640/10% FBS |
| PC3 | Prostatic adenocarcinoma | RPMI 1640/10% FBS |
| LNCaP | Prostatic adenocarcinoma | RPMI 1640/10% FBS |
| DU145 | Prostatic adenocarcinoma | MEM/10% FBS/NaP |

The human prostate LNCaP cancer cells (PTEN negative, PI3K/Akt and mTOR active) and androgen independent PC3 (PTEN negative) cells were used as a model of non-aggressive to aggressive prostate cancer. LNCaP cells are one of a few available AR-expressing and androgen-responsive prostate cancer cells that represent prostate cancer at an early stage. PC3 cells represent the late stage prostate cancer because they are androgen independent.

Anti-proliferative effect of sweet gum extract was assessed by MTT assay as described by T. Mossman, 1983. Briefly, cells (1×10⁵) were plated on 96-well plates and allowed to grow overnight. Cells were then treated with a series of concentrations of either LIS-F or LIS-100 (0.04 to 500 μg/ml) and incubated for 72 hrs. The extracts were dissolved in DMSO initially, then diluted with medium to reach a DMSO concentration of not more than 0.1% in the final treatment solution. The treated cells were then subjected to MTT assay at the end of the experimental period.

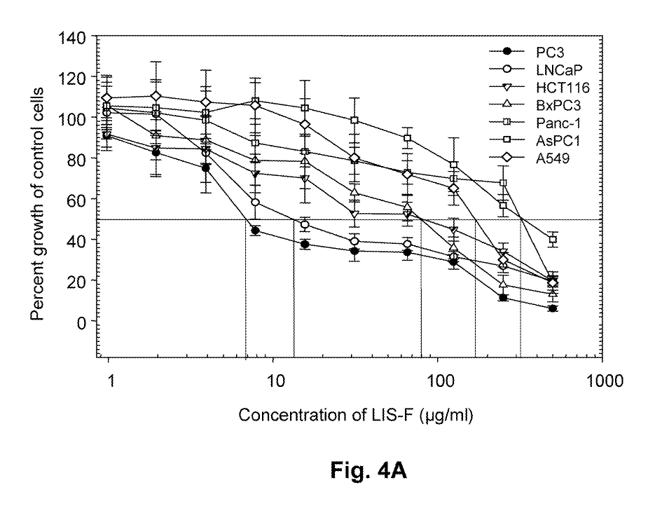

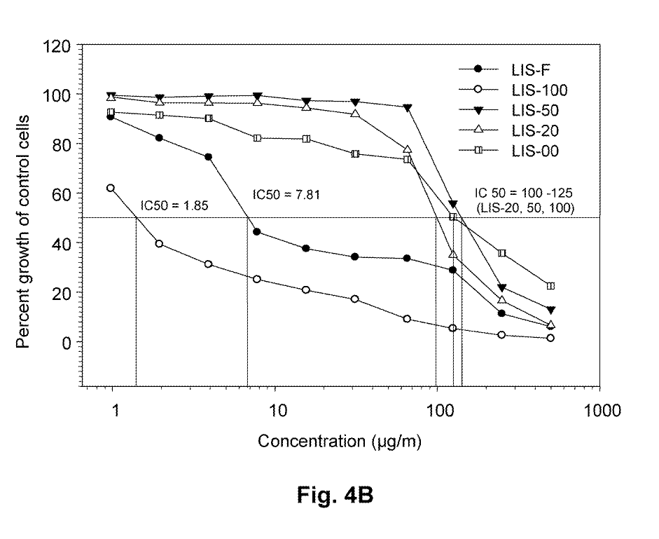

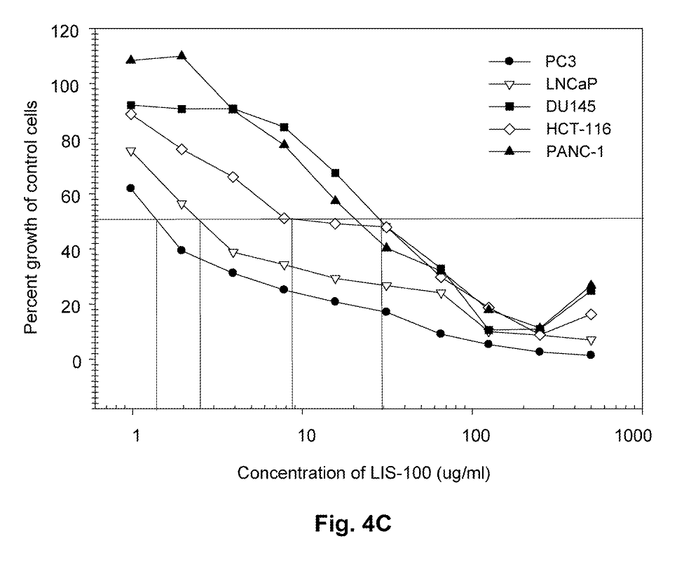

As shown in FIG. 4A, among the responses to LIS-F of the above cell lines, the inhibitory effect of LIS-F on the growth of prostate cancer cells was the most potent, with an IC₅₀ of around 7.1 μg/ml as compared with other cell lines, with IC₅₀s ranging from 80 to 122 μg/ml.

LIS-F was further characterized. One round of fractionation was performed using column chromatography based on the polarity of the components as discussed above. The effect on proliferation of human prostate PC3 cancer cells was then tested for the various fractions. As shown in FIG. 4B, anti-proliferative activity was enhanced by over 4 fold by further fractionation of LIS-F with 100% methanol (IC₅₀, 1.85 μg/ml) (LIS-100) in comparison with the activity of the crude extract (LIS-F) (IC₅₀=7.81 μg/ml). As shown in FIG. 4B, fractions eluted with 50% (LIS-50) or 20% (LIS-20) methanol were much less potent in inhibiting the proliferation of PC3 cells. LIS-00, which is a water-soluble fraction of LIS-F, did not inhibit the proliferation of PC3 cells.

When human prostate (PC3, LNCaP and DU145) cells, human colon cancer HCT116 cells, and human pancreatic Panc-1 cells were treated with LIS-100 at various concentrations, the inhibitory effect of this fraction on PC3 cells and LNCaP cells was similar, with IC₅₀s of 1.7 and 2.5 μg/ml, respectively. In comparison, LIS-100 inhibited the growth of HCT116, Panc-1, and DU145 cells by 50% at a concentration of 11.1, 21.5, and 31.2 μg/ml, respectively (FIG. 4C).

Interestingly, although DU145 prostate cancer cells are androgen independent, but PTEN (the tumor suppressor gene, a phosphatase and tensin homologue on chromosome 10) positive, the inhibitory effect of LIS-100 on that particular cell line was much weaker than that on the LNCaP and PC3 prostate cancer cells, which are PTEN negative. Without wishing to be bound by this theory, it is believed that the anticancer efficacy of LIS-100 is associated with the expression and mutation of PTEN resulting in the activation of the PI3K/Akt/mTOR pathway. The above data consistently demonstrate that prostate cancer cells are more sensitive to sweet gum extract and especially to the LIS-100 fraction than were the other malignant cells tested.

### Example 3

Characterization of the Sweet Gum Extracts

The HPLC chromatographic fingerprints of the sweet gum fruit extracts were performed as described above, and the results are shown in FIG. 5. LIS-100, a purer fraction of LIS-F, remains a complex mixture of components (FIG. 5). There are more than seventeen major peaks in the potent fraction of LIS-100. Some peaks, however, may not represent a single compound, but rather a small cluster of compounds. Compared with the crude extract (LIS), LIS-100 obviously retains the least polar compounds whereas LIS-00 retains the most polar ones, and LIS-20 and LIS-50 retain those in between.

### Example 4

LIS-100 Inhibited the Growth of PC3 Cells

LIS-F and LIS-100 extract fractions were tested for the effect on cell cycle regulation and apoptosis in PC3 cells. Cells (1×10⁶) were treated with various concentrations of either LIS-F or LIS-100 (25 to 250 μg/ml) for 24 and 48 hrs. Cells were trypsinized, washed in PBS, and fixed overnight in 70% ethanol at 4° C. They were then washed and resuspended in PBS containing 50 μg/ml of propidium iodide (PI) and 20 μg/ml of DNase-free RNase. Cells were then incubated in the dark for 30 minutes at room temperature prior to analysis by fluorescence-activated cell-sorting analysis (FACS) using a Coulter Epics-XL (Coulter Corp., Fullerton, Calif.). The percentage of cells in each phase of the cell cycle was then estimated from the DNA histogram content.

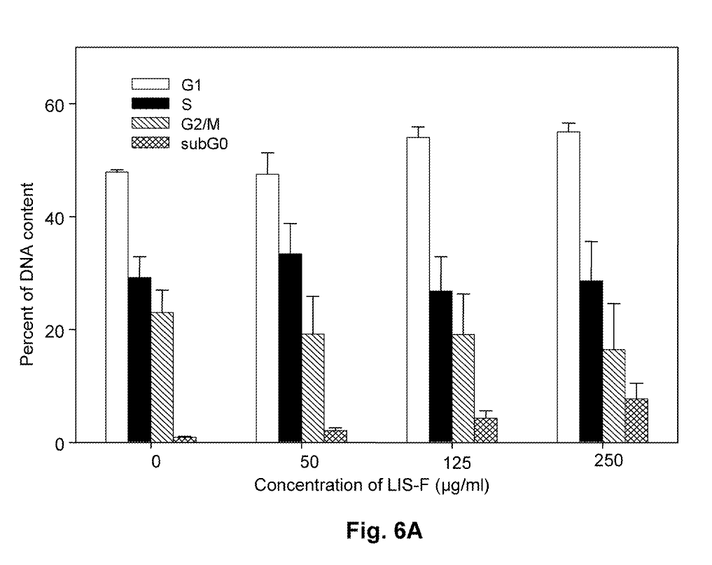

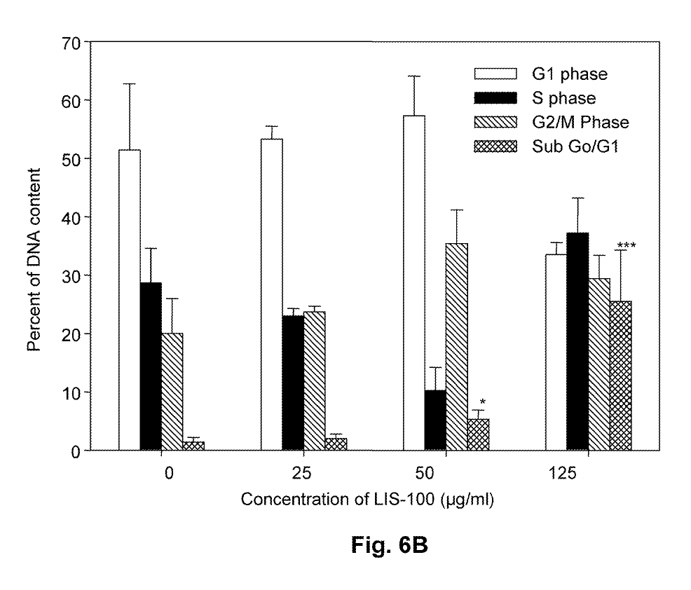

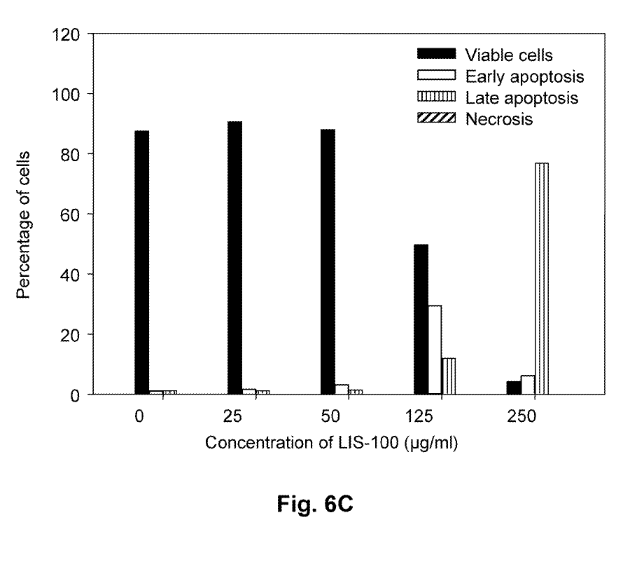

Intriguingly, when PC3 cells were treated with LIS-F (50-125 μg/ml) for 24 hrs, the growth of the cells was inhibited through G1 phase arrest in a concentration-dependent manner. (FIG. 6A) In contrast, LIS-100 at lower concentrations (25-50 μg/ml), but not higher, arrested cells in the G2/M phase, whereas apoptosis was induced in PC3 cells treated with a higher concentration (125 μg/ml) (FIG. 6B), suggesting that the two different concentrations of LIS-100 inhibited PC3 cell proliferation through different mechanisms.

These findings were confirmed by staining the LIS-100-treated PC3 cells with Annexin IV (staining for early stage of apoptotic cells) and PI (staining for late stage and necrotic cells) together (FIG. 6C), as described in Yang et al., 2003.

### Example 5

Sweet Gum Extracts Inhibition of Arachiodonk Acid Metabolites

Mediators associated with the development of prostate cancer include arachidonic acid metabolites. For this reason, the effect of LIS-F and LIS-100 on levels of eicosanoids were examined in PC3 cells as described in Yang et al., 2003. Briefly, PC3 cells were harvested by trypsinization and washed with PBS. Cells (5×10⁶) were then resuspended in 500 μl of PBS with 1 mM CaCl₂ and incubated with either LIS-F or LIS-100 (25, 50, and 100 μg/ml) for 10 min, followed by the addition of A23187, a calcium ionophore. The reaction mixture was then incubated for another 5 min, and a 5-μl aliquot of 10mM arachidonic acid solution was added and the mixture further incubated for 10 min. The reaction was stopped by the addition of 1N citric acid. Eicosanoids in the cells were then extracted and quantified with a rapid, sensitive LC/MS/MS method as described in Kempen et al., 2001 and in Yang et al., 2002. An Agilent HPLC coupled to a Quattro Ultima tandem mass spectrometer was used for these analyses.

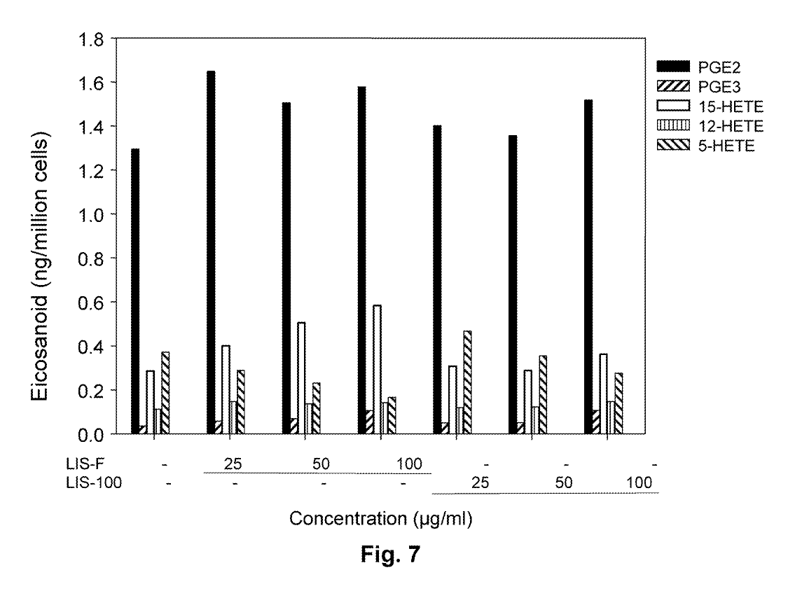

Results are shown in FIG. 7. Neither LIS-F nor LIS-100 alterned the formation of PGE₂ in PC3 cells. The most significant changes were observed in metabolites representing the lipoxygenase (LOX) pathway. Both LIS-F and LIS-100 increased the formation of 15-HETE (a 15-LOX-2 product), but inhibited the production of 5-HETE (a 5-LOX product) in a concentration-dependent manner. The change in the eicosanoid balance seen with LIS-100 was much more significant that that seen with LIS-F. The 15-LOX-2 and its arachidonate metabolite 15-HETE have been shown to function as tumor suppressors in prostate cancer cells, whereas 5-LOX and its metabolite 5-HETE are considered part of the survival pathway for prostate cancer cells. These results indicate that LIS-100 can shift the balance of arachidonate metabolism by increasing the production of the tumor suppressor 15-HETE and reducing the levels of the tumor promoter gene product 5-HETE. These alterations may play a critical role in LIS-100 mediated growth inhibition of PC3 cells.

### Example 6

LIS-100 Inhibits Multiple Cell Cycle—Associated Proteins and Cell Signaling Proteins

Studies have suggested that PTEN loss in human prostate cancer is highly correlated with the subsequent activation of the tumor-promoting protein Akt, which in turn activates mTOR and the subsequent ribosomal protein S6. Thus, the effect of LIS-F and LIS-100 on the proteins associated with the PI3K pathway, including both the PI3K/Akt and mTOR pathways, was examined in PC3 cells. PC3 cells were treated in serum-free conditions with LIS-F or LIS-100 (0.5 to 50 μg/ml) for 24 hr. Cells were then washed with cold phosphate buffered saline (PBS) and scraped with a lysis buffer, containing 20 mM MOPS, 2mM EGTA, 5 mM EDTA, 30 mM NaF, 40 mM b-glycerophosphate, 20 mM sodium pyruvate, 0.5% triton X-100, and 1 mM sodium orthovanadate with 1 × protease inhibitor cocktail (Sigma, Inc., St. Louis, Mo.). Lysates were then sonicated on ice for 3 min, incubated for 10 min, and centrifuged at (14,000 rpm) for 10 min at 4° C. Protein levels were quantified via the BioRad Dc protein assay (BioRad, Inc., Hercules, Calif.). Equal levels of protein (50 μg) were fractionated on precast gels (BioRad, Inc., Hercules, Calif.) and then transferred on polyvinylidene diflouride membranes, according to standard methods. Following a 1-2 hr incubation in 5% nonfat dry milk blocking buffer prepared in tris-buffered saline with 0.1% Tween 20 (TBS-T), membranes were probed with primary antibodies (Table 2) diluted 1:2000 in blocking buffer. Protein bands were visualized via chemiluminesence, using the ECL+ detection kit and hyper-film (Amersham Biosciences, Piscataway, N.J.). Equal loading of samples was illustrated by Western blotting for β-Actin content.

TABLE 2
Sources of primary antibodies of cell cycle and cell signaling proteins

| Name of protein | Sources of antibodies |
|---|---|
| Akt | Cell Signaling |
| pAkt | Cell Signaling |
| P70S6K | Cell Signaling |
| Pp70S6K | Cell Signaling |
| S6 | Cell Signaling |
| pS6 | Cell Signaling |
| eIF4E | AbCam |
| pIF4E | AbCam |
| Cyclin D1 | Santa Cruz or CS |
| CDK4 | BD Trans. Lab. |
| RB | Cell Signaling |
| pRB | Cell Signaling |
| elE4EBP1 | AbCam |
| β-actin | Sigma |

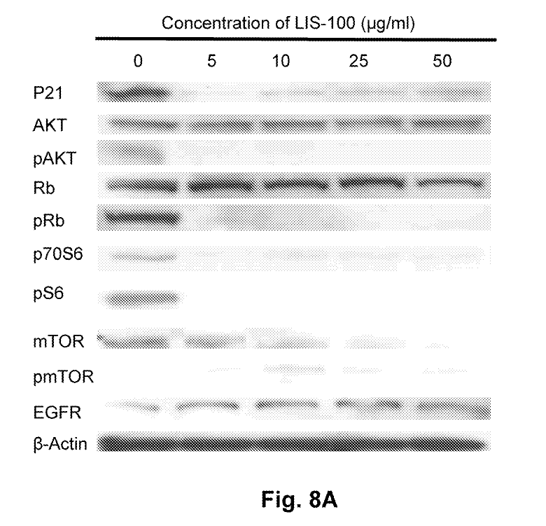

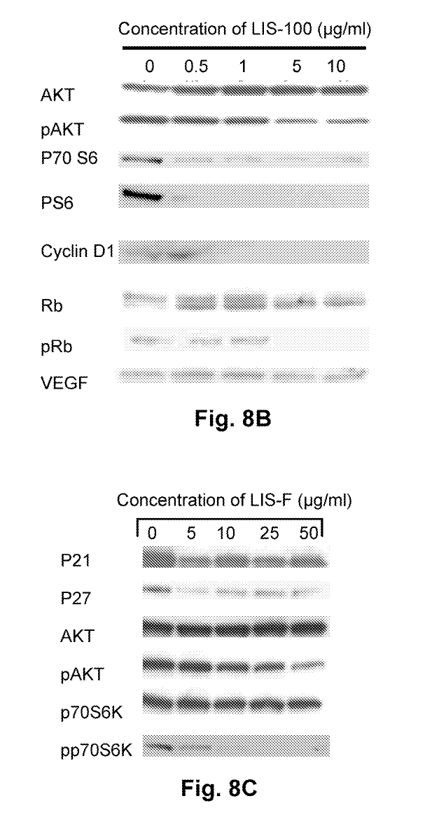

As demonstrated in FIG. 8A (Left), LIS-100 at a very low concentration (5 μg/ml) inhibited the phosphorylation of Akt, p70S6 kinase at ser389, and ribosomal S6 protein at Ser235/236. In order to confirm whether the inhibitory effect of the above proteins were concentration dependent, the levels of the above proteins in PC3 cells treated with lower concentrations of LIS-100 (0.5 to 10 μg/ml) were measured by western blotting analysis (FIG. 8B). This analysis indicated that, indeed, LIS-100 inhibited Akt phosphorylation at 5 μg/ml and the reduction was concentration dependent. In comparison to inhibition of Akt phosphorylation, LIS-100, even at 0.5 μg/ml, dramatically inhibited the phosphorylation of p70S6K and its downstream substrate S6, and this inhibition was also concentration dependent (FIG. 8B). Since the mTOR inhibitor, rapamycin, inhibits G1 cell cycle progression and the expression of cyclin D1, CDK4 and Rb phosphorylation, whether LIS-100 can cause similar biological changes was examined. As shown in FIGS. 8A and 8B, LIS-100 inhibited the expression of cyclin D 1 and the phosphorylation of the Rb protein, and the concentration of LIS-100 giving rise to those effects was very similar to the concentration that was able to inhibit the phosphorylation of p70S6K and S6. This indicates that the inhibition of PC3 proliferation by LIS-100 might predominantly be mediated through inhibition of the mTOR pathway. Because one of the pathways associated with the mTOR inhibitor, rapamycin, induced activation of Akt was through induction of IGF-I/IRS-1, the expression of phosphorylated IRS-1 in PC3 cells was examined. LIS-100 slightly reduced the phosphorylation of IRS-1 (FIG. 8A), which suggests that LIS-100 did not activate the insulin receptor while inhibiting mTOR effector proteins.

LIS-F also inhibited the phosphorylation of Akt and p70S6K in PC3 cells, but at a much higher concentration (about 10 times higher) than that of LIS-100, respectively. (FIG. 8C).

### Example 7

LIS-100 Induces Autophagic Cell Death

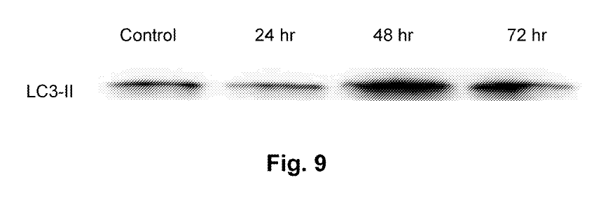

Classically, electron microscopy is used as the standard to demonstrate autophagic cell death. More recently, the autophagosome-associated protein microtubule-associated protein 1 light chain 3 (LC3) has been used as a marker for autophagy. LC3 has two forms: type I is cytosolic and type II is membrane-bound. During autophagy, LC3-II increases by conversion from LC3 type I. Therefore, upregulation of LC3 type II indicates that autophagy has occurred. In FIG. 9, cells were treated with LIS-100 (2 μg/ml) for 24, 48 and 72 hrs. At the relevant time period, cell lysates were collected and the expression of the hallmark protein of autophagy, light chain 3 protein (LC3-II), was determined by western blotting methods as described in Newman et al., 2007. Levels of LC3-II was increased after 24 hr of treatment with LIS-100 and the effect remained up to 72 hrs, suggesting LIS-100 may induce autophagic cell death in PC3 cells.

### Example 8

LIS-100 Inhibition of PI3-Kinase Activity in PC3 Cells

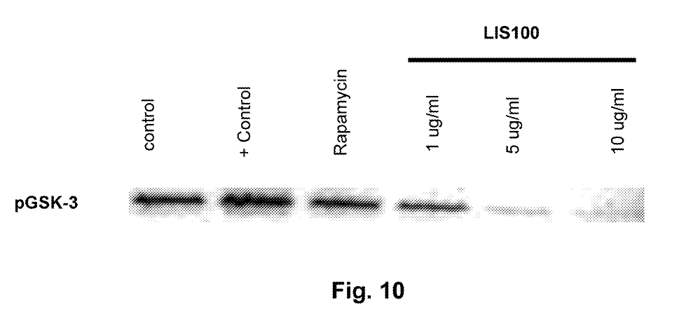

The Akt activity assay was performed using the Akt Kinase Assay Kit (Cell Signaling, Beverly, Mass.). PC3 cells were treated with various concentrations of LIS-100 (1, 5, and 10 μg/ml) for 24 hrs. The whole cell lysates were immunoprecipitated with immobilized anti-Akt antibody. The in vitro kinase assays were performed using GSK (Glycogen Synthase Kinase-3)-fusion protein as a substrate according to the manufacturer's instruction. Phosphorylation of GSK-3 was detected with anti-(phosphor-GSK-3). As shown in FIG. 10, LIS-100 significantly inhibited the activity of PI3-kinase as shown by the marked reduction of phosphorylated GSK-3. This inhibition of activity of PI3-kinase was concentration dependent.

### Example 9

LIS-100 Inhibition of mTOR Pathway in PC3 Cells

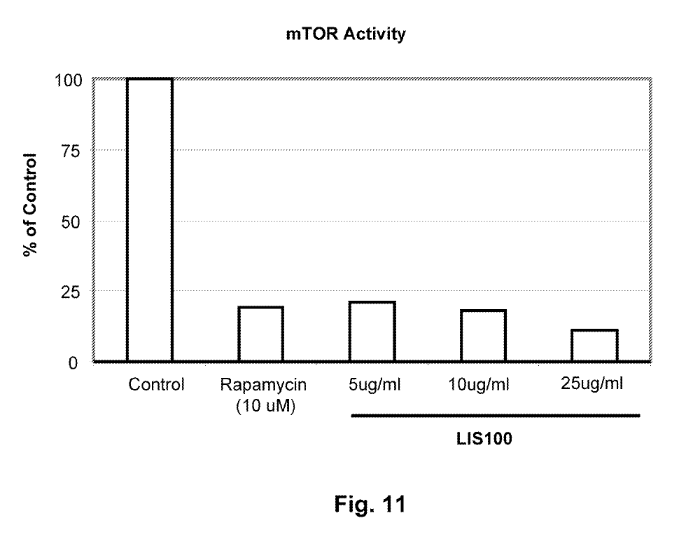

In order to confirm that LIS-100 not only inhibited the expression of the mTOR effector protein, but also down-regulated the activity of mTOR protein, PC3 cells were treated with LIS-100 at concentrations of 5, 10 and 25 μg/ml for 24 hrs. The mTOR protein was subjected to immuno-precipitation, and activity of mTOR was analyzed by an ELISA kit according to the manufacture's protocol (K-LISA mTOR Activity Kit, Calbiochem, San Diego, Calif.). A GST-fusion of P70S6K was used as a specific mTOR substrate for the in vitro mTOR activity assay. As shown in FIG. 11, LIS-100 at 5 μg/ml inhibited the activity of mTOR by almost 80%. At 10 μg/ml, the inhibitory effect of mTOR activity by LIS-100 was comparable with that produced by rapamycin (10 μM), a known mTOR inhibitor. At 25 μg/ml, the inhibition was even greater. Thus, LIS-100 inhibits the activity of mTOR and this effect is concentration dependent.

### Example 10

Acute Toxicity of LIS-100 in Mice

To check for the toxicity of LIS-100, CD-1 mice (5 per group) (Charles River Laboratory, Wilmington, Mass.) were administered with LIS-100 (DMSO: PEG 400-1:1) at the dose of 500, 100, and 50 mg/kg by either intraperitoneal (IP) injection (100 μl) or by oral gavage once per day for 5 days. Two different administration routes were selected to determine the differential bioavailability of LIS-100 in mice. Mice were monitored very carefully for the first 24 hrs, every 5 min for the first hour, every hour for the next 11 hrs and then at 16 hr and 24 hr afterward. Their weights were measured every day and clinical signs of toxicity recorded for 7 consecutive days. For the oral administration group, the body weights and behavioral observations were similar in all three groups as compared to the control group. (Data not shown) For the IP administration, an abnormal clinical observation was made with the mice dosed at 500 mg/kg, but the mice dosed at 100 and 50 mg/kg were normal. Therefore, in general, LIS-100 is considered to be relatively safe, especially if given orally.

### Example 11

LIS-100 Inhibits Prostate Cancer Tumor Growth.

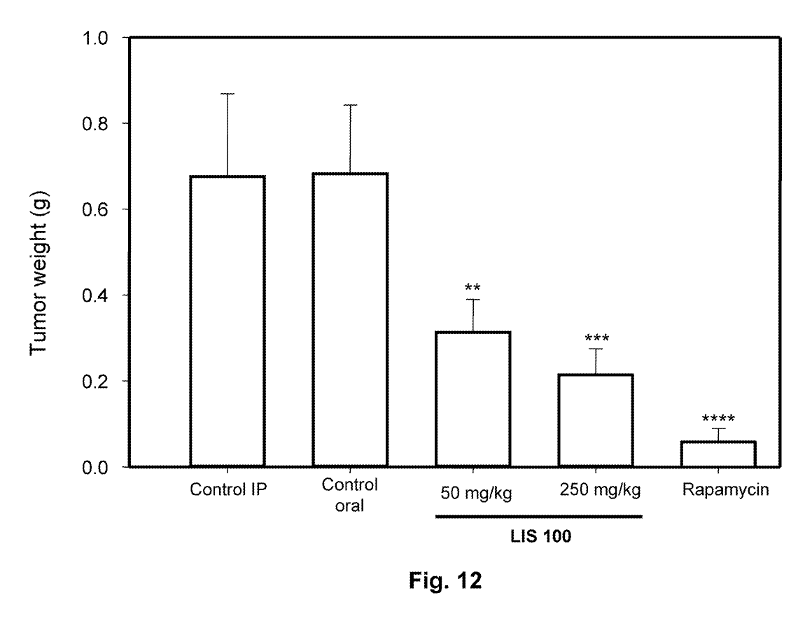

To analyze the activity of LIS-100 on tumor growth, a human prostate cancer PC3 xenograft model was used. Human prostate cancer PC3 cells (3×10⁶) were suspended in 100 μl of PBS and mixed with Matrigel (Becton Dickinson, Bedford, Mass.) (1:1). This suspension was injected subcutaneously into both flanks of Blbc/Nu/Nu mice (Charles River Laboratory). When the tumor xenografts reached 5 mm in diameter, the animals were assigned randomly into groups of 10 mice each, except for the positive group (5 mice per group). Mice were treated with LIS-100 via either intraperitoneal route (50 mg/kg) or oral gavage (250 mg/kg) once per day for 3 weeks. Control mice were given the similar amount of vehicle (DMSO: PEG 400-1:1) used for the preparation of LIS-100 in the two different routes. In addition, rapamycin (10 mg/kg per day for three weeks given by intraperitoneal injection), was used as a positive control in this experiment. The largest tumor diameter (W) and its perpendicular (L) were measured twice weekly, and the total volume was estimated by the formula, L×W×W/2. At the end, the mice were killed, and tumor specimens were collected and weighed. As shown in FIG. 12, LIS-100 significantly inhibited the growth of the tumor in this human prostate cancer PC3 xenograft model administered by oral or IP by 66% and 54%, respectively, as compared to that of the control group.

### Example 12

Further Characterization of the LIS-100 and LIS-F Extracts.

To further characterize the LIS-F and LIS-100 fractions, additional HPLC analyses were performed on a Waters 600E system with an auto sampler and a photodiode array detector. The analyses used a LUNA C18 (5 μm) column of about 250 mm×4.6 mm. The mobile phase A consisted of HPLC grade acetonitrile and mobile phase B consisted of HPLC-grade water containing 0.3% phosphoric acid. The gradient of the eluting mobile phase is described in Table 3 below. The mobile phase was pumped at 1.0 mL/min, the column temperature was 25° C., and the injection volume was 10.0 μL. The wavelength of the PDA detector ranged from 200 to 400 nm, and detections were made at 254 nm, 203 nm or 254+203 nm.

TABLE 3
Elution schemes of the HPLC analysis

| | Time | Flow | | |
|---|---|---|---|---|
| | (Min) | (ml/min) | % A | % B |
| 1 | 0 | 1.00 | 10.0 | 90.0 |
| 2 | 30.00 | 1.00 | 20.0 | 80.0 |
| 3 | 60.00 | 1.00 | 30.0 | 70.0 |
| 4 | 80.00 | 1.00 | 60.0 | 40.0 |
| 5 | 90.00 | 1.00 | 80.0 | 20.0 |
| 6 | 100.00 | 1.00 | 100.0 | 0.0 |
| 7 | 120.00 | 1.00 | 100.0 | 0.0 |

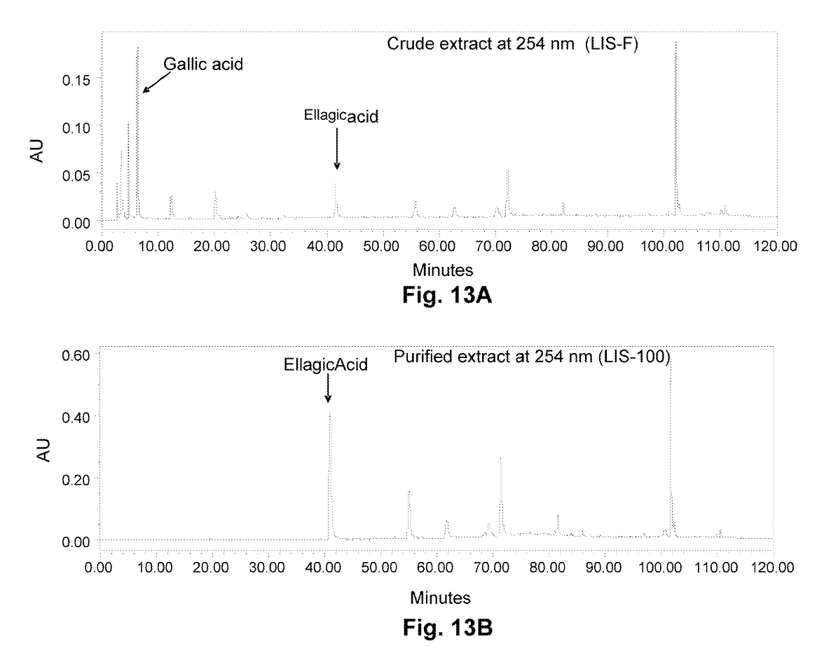

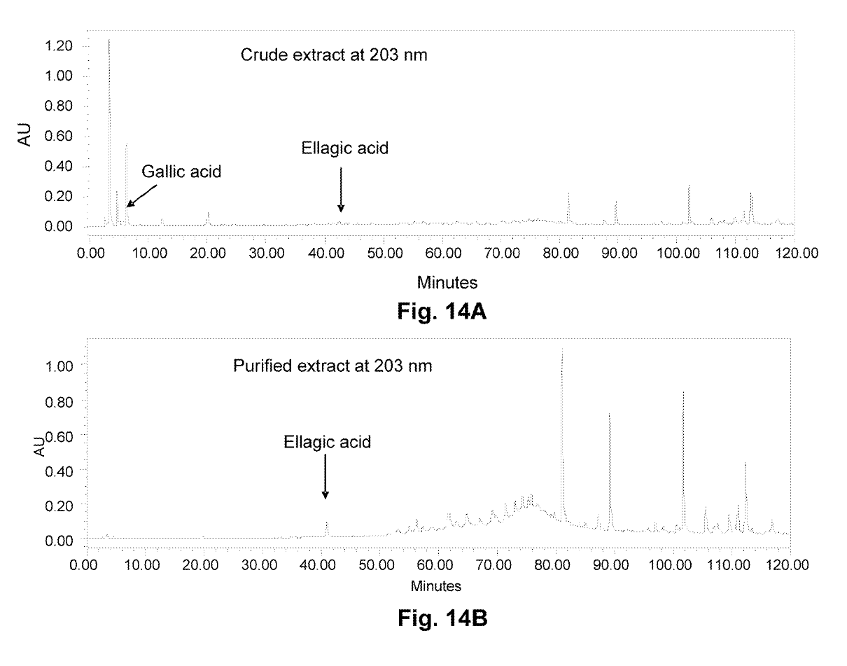

The results of the above chromatographic procedure are shown in FIGS. 13 and 14, including fingerprints of both the crude extract (LIS-F) and the purified extract (LIS-100). As shown in FIG. 13A, using 254 nm, the crude extract contained both gallic acid and ellagic acid. In contrast, FIG. 13B indicates that the purified extract (LIS-100) only had ellagic acid. In FIGS. 14A and 14B, at 203 nm, more peaks were detected between 80 and 120 min than those at 254 nm, indicative of possible locations of terpenoids. Again, as shown in FIG. 14A, the polar components such as gallic acid were removed from LIS-F to result in the purified extract (LIS-100). The purified extract contained more non-polar components such as tri-terpene glucosides, but resulting compounds have not been identified.

### Example 13

Comparison of LIS-100 and Known Components of Sweet Gum Extract

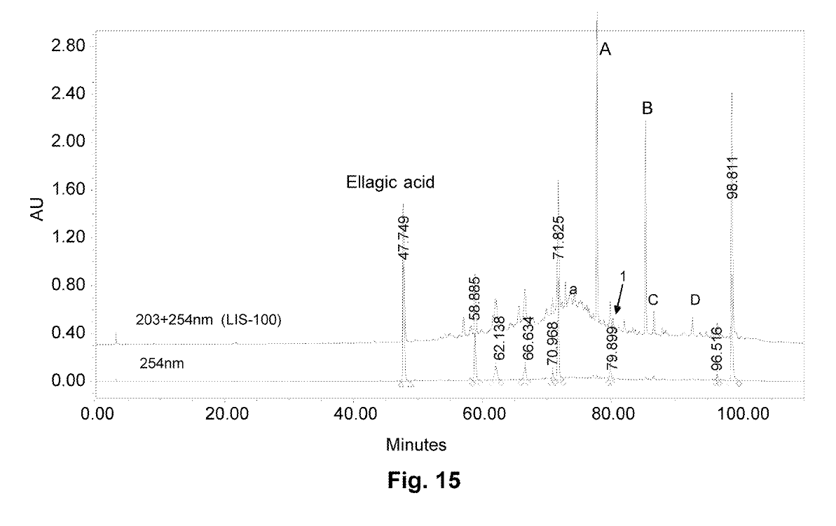

Chromatographic fingerprints of the purified LIS-100 extract was compared to a fingerprint of ellagic acid and a triterpeneoid 1 (25-acetoxy-3a-hydroxyolean-12-2n-28-oic acid, an oleanane-type triterpeneoid) that is known to occur in sweet gum extract (“Terpenoid 1”). The HPLC conditions included the use of a Prevail C18 column (about 4.6 mm×250 mm, size 5 μm). Mobile phase A consisted of HPLC grade acetonitrile, and mobile phase B consisted of HPLC-grade water containing 0.3% phosphoric acid. The gradient of the eluting mobile phase was 0-65 min at 5-30% A, 65-85 min at 30-60% A, 85-90 min at 60-70% A, and 90-100 min at 70% A. The mobile phase was pumped at 1.0 mL/min, the column temperature was 25° C., and the injection volume was 10.0 uL. The wavelength of the PDA detector ranged from 200 to 400 nm, and the detections were made at 254 nm or 254+203 nm. The results are shown in FIG. 15. Based on the results, the ellagic acid (retention time=47.749 min) content in LIS-100 is about 1.3% w/w, and the Terpeneoid 1 (retention time=80.5) is about 8.3% w/w. The peaks labeled A, B, C, and D are probably other major triterpeneoids.

Based on published articles, several triterpenoids isolated from the cones of Liquidambar styraciflua were found to be active inhibitors of tumor growth. The concentrations that induced 50% cell death (IC₅₀) of various human cancer cells in vitro ranged from 1.71 μg/ml to 17.9 μg/ml for various purified triterpenoids compounds. In comparison, the concentration of LIS-100 that induced 50% cell death (IC₅₀) ranged from 1.85 μg/ml to 31.2 μg/ml for multiple cancer cell lines tested. In particular, the IC₅₀ value of the reported Terpenoid 1 against human prostate cancer PC3 cells was 5.9 μg/ml. This is in contrast to only 1.85 μg/ml of the sweet gum extract LIS-100 to exert the same effect against PC3 cells. It is believed that the high potency of LIS-100 is due to the dual inhibition of PI3-kinase and mTOR pathways, a synergistic approach to treating cancer. (Fukuda et al., 2006; Sakai et al., 2004)

LC-MS analysis of LIS-100 extract indicated that Terpeneoid 1 (25-acetoxy-3α-hydroxyolean-12-en-28-oic acid, an oleanane-type triterpeneoid) was present in an amount of about 8.3% w/w. Based on this analysis and the observed IC₅₀ in various cancer cells treated with the Liquidambar styraciflua fruit extract (LIS-100) that ranged from 1.85 μg/ml to 31.25 μg/ml, at these IC₅₀ concentration, Terpenoid 1 would be present at concentrations about 0.15 μg/ml to 2.59 μg/ml. Clearly, the effectiveness of LIS-100 is greater than Terpeneoid 1 alone, and shows additive and synergistic activity that make it 15 times more effective than Terpeneoid 1.

As shown above, the sweet gum extract, especially its methanolic fraction, has been shown to inhibit the proliferation of multiple cell lines with IC50 values ranging from 1.85 to 31.25 μg/ml and was most potent against prostate cancer (IC50 of 1.85 and 2.75 μg/ml for PC3 and LNCaP cells, respectively). LIS-100 inhibited the proliferation of PC3 cells through G2/M cell cycle arrest at low concentrations (10 to 50 μg/ml), but induced apoptosis at higher concentrations (about 125 μg/ml). It inhibited cyclin D1 expression, Stat3 and restored the tumor suppressor Rb protein function in PC3 cells. Additionally, LIS-100 (at 5 μg/ml) altered the phosphorylation of multiple cell signaling proteins, especially those associated with PI3K/Akt/mTOR pathways. LIS-100 not only inhibited phosphorylation of the effector of mTOR, p70S6 kinase at ser389, and ribosomal S6 protein at Ser235/236, it also inhibited the phosphorylation of Akt and eIF4E in both PC3 and LNCaP cells, yet increased the tumor suppressor protein eIF4BP1. These above effects were concentration dependent. In comparison to a known mTOR inhibitor (a rapamycin derivative) which enchances cell survival pathways, such as PI3K-Akt and 4IF4E, the LIS-100 extract has the ability to inhibit both PI3K/Akt and mTOR pathways. When the LIS-100 extract was administered orally to CD-1 mice, no signs of toxicity were observed even at does as high as 500 mg/kg. Additionally, the LIS-100 extract inhibited the growth of PC3 xenograft by 66% and 54%, when administered at 250 mg/kg orally and 50 mg/kg intraperitoneally, respectively. The most striking result, is that LIS-100, as a plant extract, at a very low concentration can strongly inhibit the mTOR pathway while simultaneously inhibiting the phosphorylation of Akt. This dual inhibition is especially important for treatment of the particularly aggressive form of prostate cancer.

Miscellaneous

The term “active sweet gum fruit extract” is defined as an extract from the fruit of sweet gum (Liquidambar styraciflua L., family Hamamelidacease) that possesses inhibitory activity against multiple targets of the PI3K (phosphatidylinositide 3-kinase) pathway, especially the PI3K/Akt and mTOR pathways. An example of an active sweet gum fruit extract is LIS-100 which has the chromatographic profile as in FIGS. 5, 13B, 14B, and 15. For convenience, in the Claims, the extract will be identified as the chromatogram in FIG. 15.

The term “effective amount” as used herein refers to an amount of “active sweet gum fruit extract” sufficient to inhibit the PI3K/Akt and mTOR pathway to a statistically significant degree (p<0.05). The term “effective amount” therefore includes, for example, an amount sufficient to decrease the growth of late stage prostate cancer or other forms of cancer, preferably by at least 50%, and more preferably to by at least 90%. The dosage ranges for the administration of active sweet gum fruit extract are those that produce the desired effect. Generally, the dosage will vary with the age, weight, condition, sex of the patient, type of tumor or other pathology, and the degree of tumor development. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the growth of the tumor by methods well known to those in the field. Moreover, active sweet gum fruit extract can be applied in pharmaceutically acceptable carriers known in the art. Active sweet gum fruit extract can be used to treat cancers in animals and in humans in vivo. The application can be oral, by injection, or topical, providing that in an oral administration active sweet gum fruit extract is preferably protected from digestion.

Active sweet gum fruit extract may be administered to a patient by any suitable means, including oral, parenteral, subcutaneous, intrapulmonary, topical, and intranasal administration. Parenteral infusions include intramuscular, intravenous, intraarterial, intraperitoneal or intravitreal administration. Additionally, the infusion could be into an organ or tumor or site of disease. Injection of active sweet gum fruit extract may include the above infusions or may include intraperitonieal, intravitreal, or direct injection into a tumor. Active sweet gum fruit extract may also be administered transdermally, for example in the form of a slow-release subcutaneous implant, or orally in the form of capsules, powders, or granules. Although direct oral administration may cause some loss of anti-tumorigenic activity, active sweet gum fruit extract could be packaged in such a way to protect the active ingredient(s) from digestion by use of enteric coatings, capsules or other methods known in the art.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, and glycerol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampule, each containing a unit dose amount, or in the form of a container containing multiple doses. Direct injections into a tumor tissue or fat mass would be the most direct way to deliver the active sweet gum fruit extract to the target tissue.

Active sweet gum fruit extract may be formulated into therapeutic compositions as pharmaceutically acceptable salts. These salts include the acid addition salts formed with inorganic acids such as, for example, hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

Controlled delivery may be achieved by admixing the active ingredient with appropriate macromolecules, for example, polyesters, polyamino acids, polyvinyl pyrrolidone, ethylenevinylacetate, methylcellulose, carboxymethylcellulose, prolamine sulfate, or lactide/glycolide copolymers. The rate of release of active sweet gum fruit extract may be controlled by altering the concentration of the macromolecule.

Controlled delivery can also be achieved by conjugating active sweet gum fruit extract with a known compound that targets cellular surface receptors that are known to be unique to specific tumor types.

Another method for controlling the duration of action comprises incorporating active sweet gum fruit extract into particles of a polymeric substance such as a polyester, peptide, hydrogel, polylactide/glycolide copolymer, or ethylenevinylacetate copolymers. Alternatively, active sweet gum fruit extract may be encapsulated in microcapsules prepared, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

The present invention provides a method of treating or ameliorating a disease that would be inhibited by compounds with activity against the PI3K/Akt and mTOR pathways such as late stage prostate cancer, other forms of cancer, diabetes, or obesity, comprising administering to a subject at risk for a disease or displaying symptoms for such disease, a therapeutically effective amount of active sweet gum fruit extract. The term “ameliorate” refers to a decrease or lessening of the symptoms or signs of the disorder being treated. The symptoms or signs that may be ameliorated include those associated with an increase in tumor growth, or symptoms of diabetes (e.g., blood glucose), or change in body weight. The term “substantially similar” is understood by a person skilled in the art to refer to the possibility that small differences in factors and concentrations of factors may exist between two compositions even after following the same extraction procedure, but that these small differences do not affect the properties as measured in the original composition.

### REFERENCES

Briaud, I., et al. Insulin receptor substrate-2 proteasomal degradation mediated by a mammalian target of rapamycin and amino acids in adipogenesis, Diabetes 53: 2748-2756, 2005.
Chambard, J. C., Lefloch, R., Pouyssegur, J., Lenormand, P. ERK implication in cell cycle regulation. Biochim. Biophys. Acta 17, 2006.
Chow, L,. M, Baker, S. J. PETN function in normal and neoplastic growth. Cancer Lett. 241: 184-196, 2006.
Cully, M. You, H., Levine, A. J., Mak, T. W. Beyond PTEN mutations: the PI3K pathways as an integrator of multiple inputs during tumorigenesis. Nat. Rev. cancer. 6: 184-192, 2006.
Fan, Q., Knight, Z. A., Goldenberg, D. D., Yu, W., Mostov, K. E., Stokoe, D., Shokat, K. M., and Weiss, W. A. A dual PI3-kinase/mTOR inhibitor reveals emergent efficacy in glioma. Cancer Cell 9: 341-349, 2006.
Fingar, D. C. and Blenis, J. Target of rapamycin (TOR): an integrator of nutrient and growth factor signals and coordinator of cell growth and cell cycle progression. Oncogene 23: 3151-3171, 2004.
Fukuda, Y., Sakai, K., Matsunaga, S., Tokuda, H. and Tanaka, R. Cancer chemopreventive activity of lupine- and oleanane-type triterpenoids from the cones of Liquidambar styraciflua. Chem Biodivers 2: 421-8, 2005.
Fukuda, Y., Sakai, K., Matsunaga, S., Tokuda, H. and Tanaka, R. Cancer chemopreventive effect of orally administrated lupine-type triterpenoid on ultraviolet light B induced photocarcinogenesis of hairless mouse. Cancer Lett. 240 : 94-101, 2006.
Fukuda, Y., Yamada, T., Wada, S. I., Sakai, K., Matsunaga, S., and Tanaka, R. Lupane and oleanane triterpenoids from the cones of Liquidambar styraciflua. J. Nat. Prod. 69: 142-4, 2006.
Gao, N., Zhang, Z., Jiang, B., and Shi, X. Role of PI3K/Akt/mTOR signaling in the cell cycle progression of human prostate cancer. Biochem. Biophy. Res. Communi. 310: 1124-1132, 2003.
Harrington, L. S., et al. The TSC1-2 tumor suppressor controls insulin-PI3K signaling via regulation of IRS protein. J. Cell Biol. 166: 213-223, 2004.
Hartford, C. M. et al. Rapamycin: Something old, something new, sometimes borrowed and now renewed. Clinical Pharmacology & Therapeutics, 82:381-388.
Higgs, G. A., Moncada, S., and Vane J. R. Eicosanoids in inflammation. Ann. Clin. Res. 16: 287-299, 1984.
Jiang., M., Shappell, S. B., and Hayward, S. W. Approaches to inderstanding the importance and clinical implications of peroxisome proliferator-activated receptor Gamma (PPARγ) signaling in prostate cancer. J. Cell Biol. 91 : 513-527, 2004.
Jordan, S. Comparison of American and oriental storax. Journal of Industrial and Engineering Chemistry (Washington, D.C.), 9: 770-1, 1919.
Kempen, E. C. et al., Simultaneous quantification of arachidonic acid metabolites in cultured tumor cells using high performance liquid chromatography/electrospray ionization tandem mass spectrometry. Anal. Biochem. 297:183-190, 2001.
Le Bacquer, O. et al. Elevated sensitivity to diet-induced obesity and insulin resistance to mice lacking 4E-BPI and 4E-BP2. J. Clin. Invest. 117:387-396, 2007.
McCarty, M. F. Targeting multiple signaling pathways as a strategy for managing prostate cancer:multifocal signal modulation therapy. Interg. Cancer Therap. 3 : 349-380, 2004.
Mossman, T. Rapid colorimetric assay for cellular growth and survuval: application to proliferation and cytotoxicity assays. J. Immun. Methods. 65: 55-63, 1983.
Nakajima, S., Takeda, T., Nakahira, K. Nitoda, T., and Baba, N. Insect-neuroactive substances in two species of the genus Liquidambar. Pesticide Science 55: 205-206, 1999.
Nie, D., Che, M., Grignon, D., Tang, K., and Honn, K. V. Role of eicosaniods in prostate cancer progression. Cancer Metastasis Rev. 20: 195-206, 2001.
Nie, D., Nemeth, J., Qiao, Y., Zacharek, A., Li, L., Hanna, K., Tang, K., Hillman, G. G., Cher, M. L., Grigon, D. J., and Honn, K. V. Increased metastatic potential in human prostate carcinoma cells by overexpression of arachidonate 12-lipoxygenase. Clin. Exp. Metastasis 20: 657-663, 2003.
O'Reilly, K. E., Rojo, F., She, Q., Solit, D., Mills, D., Smith, D., Lane, H., Hofmann, F., Hicklin, D. J., Ludwig, D. L., Baselga, J., and Rosen, N. mTOR inhibition induces upstream receptor tyrosine kinase signaling and activates Akt. Cancer Res. 66: 1500-1508, 2006.
Sakaguchi, M., et al. Inhibition of mTOR signaling with rapamycin attenuates renal hypertrophy in the early diabetic mice. Biochem. Biophys. Res. Comm. 340: 296-301, 2006.
Sakai, K., Fukuda, Y., Matsunaga, S., Tanaka, R., and Yamori, T. New cytotoxic oleanane-type triterpenoids from the cones of Liquidambar styraciflua. J. Nat. Prod. 67: 1088-93, 2004.
Saltie, A. amd Kahn, C. Insulin signaling and the regulation of glucose and lipid metabolism. Nature 414: 799-806, 2001.
Samuels, Y., Ericson, K. Oncogenic PI3K and its role in Cancer. Curr. Opin. Oncol. 18: 77-82, 2006.
Sarbassov, D. D, Ali, S. M., Sabatini, D. M. Growing roles for the mTOR pathways. Curr. Opin. Cell Biol. 17: 596-603, 2005.
Shah, O., et al. Inappropriate activation of the TSC/Rheb/mTOR/S6K cassett induces IRS1/2 depletion, insulin resistance, and cell survival deficiencies. Curr. Biol. 14: 1650-1656, 2004.
Shapiro, A. M. J. et al. Islet transplantation in seven patients with type 1 diabetes mellitus using a glucocoriticoid-free immunosuppressive regimen. N. Eng. J. Med. 343: 230-238, 2000.
Shi, Y., Yan, H., Frost, P., Gera, J. and Lichtenstein, A. Mammalian target of rapamycin inhibitors activate the Akt kinase in multiple myeloma cells by upregulating the insulin-like growth factor receptor/insulin receptor substrate-1/phosphatidylinositol 3-kinase cascade. Mol. Cancer Ther. 4: 1533-1540, 2005.
Smolewski, P. Investigating mammalian target of rapamycin inhibitors for their anticancer properties. Expert. Opin. Investg. Drug 15: 1201-1227, 2006.
Subbarayan V., Sabichi, A. L. Llansa, Lippman, S. M. and Menter, D. G. Differential expression of cyclooxygenase-2 and its regulation by tumor necrosis factor-α in normal and malignant prostate cells. Cancer Res. 61 : 2720-2726, 2001.
Sun, S., Rosenberg, L. M., Wang, X., Zhou, Z., Yue, P., Fu, H., and Khuri, F. R. Activation of Akt and e1F4E survival pathwys by rapamycin-mediated mammalian target of rapamycin inhibition. Cancer Res. 65: 7052-7058, 2005.
Tang S, Bhatia B, Maldonado C J, Yang P, Newman R A Et al. Evidence that arachidonic acid 15-lipoxygenase-2, whose expression is lost in prostate cancer cells, is a negative cell-cycle regulator in normal prostate epithelial cells. J Biol Chem 277: 16189-201, 2002.
Testa, J. R., and Bellacosa A., AKt plays a central role in tumorigenesis. Proc. Natl. Acad. Sci. USA 98: 10983-10985. 2001.
Tortora, G, Caputo, R, Daminano, V, et al. Combination of a selectives cyclooxygenase-2 inhibitor with epidermal growth factor receptor tyrosine kinase inhibitor ZD1839 and protein kinase A antisense causes cooperative antitumor and antiangiogenic effect. Clin. Cancer Res. 9 : 1380-1572, 2003.
Tsang, C. K. et al. Targeting mammalian target of rapamycin (mTOR) for health and diseases. Drug Discovery Today 12:112-124, 2007.
Vignot, S., Faivre, S., Aguirre, D., and Raymond, E. mTOR-targeted therapy of cancer with rapamycin derivatives. Ann. Oncol. 1-13, 2005.
Wallace, J. M. Nutritional and botanical modulation of the inflammatory cascade-eicosanoids, cyclooxygenases, lipoxygenases—as an adjunt in cancer therapy. Inter. Cancer Therap. 1: 7-37, 2002.
Wang, Y., Corr, J. G., Thaler, H. T., Tao, Y., Fair, W. R., and Heston, W. D. Decreased growth of established human prostate LNCaP tumors in nude mice fed a low-fat diet. J. Nat. Cancer Inst. 87: 1456-1462.
Xu, Y., Chen, S., Ross, K. N., and Balk, S. P. Androgens induce prostate cancer cell proliferation through mammalian target of rapamycin activation and post-transcriptional increases in cyclin D proteins. Cancer Res. 66: 7783-7792, 2006.
Yang, P, Collin, P, Madden, T, Chan, D, Sweeney-Gotsch, B, McConkey, D, and Newman R A Inhibition of proliferation of PC3 cells by the branched-chain fatty acid, 12-methyltetradecanoic acid, is associated with inhibition of 5-lipoxygenase. The prostate 55: 281-291, 2003.
Yang, P. et al. Quantitative high performance liquid chromatography/electrospray ionization tandem mass spectrometric analysis of 2- and 3-series prostaglandins in cultured tumor cells. Anal. Biochem. 308:168-177, 2002.
Yang, P., Cartwright, C., Chan, D., V. M., Ding, J. and Newman, R. A. Zyflamend-Mediated Inhibition of Human Prostate Cancer PC3 Cell Proliferation: Effects on 12-LOX and Rb Protein Phosphorylation. Cancer Biol. Ther. 6: 2007.

The complete disclosures of all references cited in this application are hereby incorporated by reference. Also incorporated by reference is the complete disclosure of the following: P. Yang et al., “Sweet gum extract inhibits proliferation of prostate cancer through dual inhibition of PI3K/Akt and mTOR pathways,” an abstract and presentation to the American Association for Cancer Research Annual Meeting, Apr. 12-16, 2008, San Diego, Calif. In the event of an otherwise irreconcilable conflict, however, the present specification shall control.

What is claimed:

1. A method of treating a non-cancer disorder in a mammalian patient by inhibiting the mTOR pathway, said method comprising administering to the patient an effective amount of active sweet gum fruit extract.

2. The method of claim 1, wherein the non-cancer disorder is selected from the group consisting of rejection of allografts, autoimmune disorders, allergic encephalomyelitis, insulin-dependent diabetes mellitus, lupus, adjuvant arthritis, rheumatoid arthritis, psoriasis, and multiple sclerosis, diabetes, obesity, cardiovascular disease, ischemic disease, hypertension, valvular disease, and heart failure, and neurological disorders Huntington's disease, Alzheimer's disease and Parkinson's disease.

3. The method of claim 1, wherein the non-cancer disorder is diabetes.

4. The method of claim 1, wherein the non-cancer disorder is obesity.

5. A method of treating late-stage prostate cancer in a mammalian patient, said method comprising administering to the patient an effective amount of an active sweet gum fruit extract.

6. A composition of a purified fraction of a sweet gum fruit extract, wherein said composition is more soluble in alcohol than in water; wherein said composition contains less than about 2% w/w ellagic acid; wherein said composition contains less than about 10% w/w 25-acetoxy-3a-hydroxyolean-12-en-28-oic acid; wherein said composition contains at least one other triterpenoid; wherein said composition inhibits mTOR; and wherein said composition has a chemical fingerprint on high performance liquid chromatography at 203 and 254 nm substantially as shown in FIG. 15.

7. A method of treating a non-cancer disorder in a mammalian patient by inhibiting mTOR, said method comprising administering to the patient an effective amount of the active sweet gum fruit extract in claim 6.

8. The method of claim 7, wherein the non-cancer disorder is selected from the group consisting of rejection of allografts, autoimmune disorders, allergic encephalomyelitis, insulin-dependent diabetes mellitus, lupus, adjuvant arthritis, rheumatoid arthritis, psoriasis, and multiple sclerosis, diabetes, obesity, cardiovascular disease, ischemic disease, hypertension, valvular disease, and heart failure, and neurological disorders Huntington's disease, Alzheimer's disease and Parkinson's disease.

9. The method of claim 8, wherein the non-cancer disorder is diabetes.

10. The method of claim 8, wherein the non-cancer disorder is obesity.

11. A method of treating cancer in a mammalian patient, said method comprising administering to the patient an effective amount of the active sweet gum fruit extract as in claim 6.

12. The method of claim 11, wherein said cancer is selected from the group consisting of prostate cancer, renal cell carcinoma, mantle cell lymphoma, endometric cancers, and other cancers treatable by mTOR inhibitor.

13. A method of claim 11, wherein the cancer is prostate cancer.

14. The method of claim 13, wherein the prostate cancer is late-stage prostate cancer.

15. A method of treating in a mammal a disease that activates the PI3K/Akt and mTOR pathways, said method comprising administering to the mammal a therapeutically effective amount of active sweet gum fruit extract.

16. The method of claim 15, wherein said active sweet gum fruit extract is as in claim 6.
